(19) United States
(12) Patent Application Publication
(10) Pub. No.: US 2018/0003676 A1
(43) Pub. Date: Jan. 4, 2018

(54) MAGNETIC NANOPARTICLE, HAVING A CURIE TEMPERATURE WHICH IS WITHIN BIOCOMPATIBLE TEMPERATURE RANGE, AND METHOD FOR PREPARING SAME

(71) Applicant: KOREA UNIVERSITY RESEARCH AND BUSINESS FOUNDATION, Seoul (KR)

(72) Inventors: Young Keun KIM, Seoul (KR); Jun Hua WU, Seoul (KR); Ji Hyun MIN, Seoul (KR); Ji-Sung LEE, Seoul (KR); Jae-Ho JUNG, Gyeonggi-do (KR)

(21) Appl. No.: 15/702,062

(22) Filed: Sep. 12, 2017

Related U.S. Application Data
(62) Division of application No. 13/982,819, filed on Jul. 31, 2013.

(30) Foreign Application Priority Data
Jan. 31, 2011 (KR) 10-2011-0009824

(51) Int. Cl. G01N 27/72; C01G 45/12; G01N 33/58; A61K 49/18

(52) U.S. Cl. CPC G01N 27/72; A61K 49/1851; A61K 49/1866; A61K 49/1875; C01G 45/12; G01N 33/587; A61K 49/1827; C01P 2002/72; C01P 2004/04; C01P 2004/62; C01P 2004/64; C01P 2006/42; C01P 2002/85

(57) ABSTRACT

The present invention relates to a magnetic nanoparticle having a Curie temperature which is within a biocompatible temperature range, a method for preparing same, and a nanocomposite and a target substance detection composition comprising the magnetic nanoparticle. As the magnetic nanoparticle of the present invention has a Curie temperature within the temperature range of 0 degrees centigrade to 41 degrees centigrade, the ferromagnetic and paramagnetic properties of the magnetic nanoparticle may be controlled within a biocompatible temperature range at a temperature at which a biological control agent is not destroyed, and the temperature of the magnetic nanoparticle is adjusted to control the magnetic properties thereof such that the properties of the magnetic nanoparticle may be used only when ferromagnetic properties are required, such as in the case of signal amplification in detecting, separating, and delivering biological control agents. Accordingly, the magnetic nanoparticle of the present invention can minimize adverse effects of ferromagnetic properties thereof, and can be used in the effective detection and separation of biological control agents.

### CROSS-REFERENCE TO RELATED APPLICATION

This application is a divisional application of U.S. application Ser. No. 13/982,819, filed Jul. 31, 2013.

### BACKGROUND

### 1. Field of the Invention

The present invention relates to magnetic nanoparticles having a Curie temperature within a biocompatible temperature range, methods for preparing the same, nanocomposites for target substance detection comprising the same, and methods for obtaining an image of a living body or specimen.

### 2. Discussion of Related Art

Detection of a biological control agent using a magnetic nanoparticle is easy to use and causes relatively less damage to a detected cell, and thus is a subject of much interest. Recent studies have aimed to increase a magnetization value of a magnetic nanoparticle in order to increase sensitivity of biological control agent detection based on magnetic properties. In addition to a detection apparatus using a magnetic nanoparticle, a detection apparatus using biotin-avidin bonding is often used for biological control agent detection, and signal amplification, yet this detection apparatus has many non-specific responses and high signal noise.

Meanwhile, the most problematic issue in applying a magnetic nanoparticle in the domain of bio-medical technology is agglomeration of the magnetic nanoparticles. When the magnetic nanoparticle is used in a living body, agglomeration causes precipitation in blood vessels, triggering thrombosis, and thereby the magnetic nanoparticle's outer surface area decreases and efficiency of a magnetic nano-based diagnosis/drug delivery/medicine may decrease. In particular, in a case of a diagnosis system based on a magnetic nanoparticle used in vitro, agglomeration of the magnetic nanoparticles interferes with biochemical reactions such as an antigen-antibody reaction, causing an increase in signal noise, and thus diagnosis efficiency may decrease.

Accordingly, there is a great need to develop a magnetic nanoparticle which can decrease non-specific responses and increase signal detection sensitivity, thereby increasing a ratio of signal to noise (signal purification).

### SUMMARY OF THE INVENTION

The present invention is directed to providing magnetic nanoparticles having a Curie temperature within a biocompatible temperature range, methods for preparing the same, nanocomposites and compositions for target substance detection comprising the same, and methods for obtaining an image of a living body or specimen.

One aspect of the present invention provides a magnetic nanoparticle having a Curie temperature within the temperature range of −80° C. to 41° C., comprising a rare earth metal, a divalent metal, and a transition metal oxide.

Another aspect of the present invention provides a method for preparing a magnetic nanoparticle according to the present invention, comprising (a) a step of reducing a precursor of the rare earth metal, a precursor of the divalent metal, and a precursor of the transition metal oxide, thereby forming the magnetic nanoparticle; and (b) a step of heat treating the magnetic nanoparticle.

Still another aspect of the present invention provides a nanocomposite comprising a magnetic nanoparticle according to the present invention; and a biological control agent attached to a surface of the magnetic nanoparticle.

Yet another aspect of the present invention provides a composition for target substance detection comprising a magnetic nanoparticle according to the present invention or a nanocomposite according to the present invention; and a magnet-antibody composite.

A further aspect of the present invention provides a method for obtaining an image of a living body or specimen, comprising a step of administering a composition for target substance detection according to the present invention to a living body or specimen; and a step of sensing a signal transmitted by a magnetic nanoparticle or nanocomposite from the living body or specimen, thereby obtaining the image.

### BRIEF DESCRIPTION OF THE DRAWINGS

FIG. 1 illustrates a nanocomposite according to one embodiment of the present invention.

FIG. 2 illustrates a magnetic nanoparticle according to the present invention to which a detection means is attached.

FIG. 3 illustrates a nanocomposite according to another embodiment of the present invention to which a detection means is attached.

FIG. 4 is a schematic diagram showing a process of detecting a target substance using a composition for target substance detection according to one embodiment of the present invention.

FIG. 5 is a picture of a high-resolution transmission electron microscope (TEM) of a magnetic nanoparticle according to one embodiment of the present invention.

FIG. 6 is a graph of a X-ray diffraction (XRD) pattern of a magnetic nanoparticle according to one embodiment of the present invention.

FIG. 7 is a graph of magnetization value versus temperature (M-T) of a magnetic nanoparticle according to one embodiment of the present invention.

### BRIEF DESCRIPTION OF ELEMENTS IN THE DRAWINGS

1: nanocomposite/10: magnetic nanoparticle

11: biological control agent/12: detection means

20: substrate/21: target substance

22: impurities/23: antibody

24: magnet/27: massive composite

### DETAILED DESCRIPTION OF EXEMPLARY EMBODIMENTS

Hereinafter, exemplary embodiments of the present invention will be described in detail. However, the present invention is not limited to the exemplary embodiments disclosed below, but can be implemented in various forms. The following exemplary embodiments are described in order to enable those of ordinary skill in the art to embody and practice the invention.

It will be understood that, although the terms first, second, etc. may be used herein to describe various elements, these elements should not be limited by these terms. These terms are only used to distinguish one element from another. For example, a first element could be termed a second element, and similarly, a second element could be termed a first element, without departing from the scope of the present invention. As used here, the term “and/or” includes any and all combinations of one or more of the associated listed items.

The present invention concerns magnetic nanoparticles having a Curie temperature within the temperature range of −80° C. to 41° C., comprising a rare earth metal, a divalent metal, and a transition metal oxide.

Hereinafter, a magnetic nanoparticle of the present invention will be described in detail.

A magnetic nanoparticle according to the present invention has a Curie temperature within the temperature range of −80° C. to 41° C., preferably the temperature range of 0° C. to 41° C., and more preferably the temperature range of 10° C. to 41° C., comprising a rare earth metal, a divalent metal, and a transition metal oxide.

In the present invention, an expression “Curie temperature” may denote a critical temperature where a ferromagnet loses its magnetic properties due to an increase in temperature, and the ferromagnet exhibits paramagnetic properties at a temperature equal to and above the Curie temperature.

As the magnetic nanoparticle of the present invention has a Curie temperature within a biocompatible temperature range, ferromagnetic and paramagnetic properties of the magnetic nanoparticle may be controlled within a temperature range at which a biological control agent is not destroyed.

An average diameter of a magnetic nanoparticle according to the present invention is not particularly limited, and may be, for instance, 1 nm to 500 nm, preferably 10 nm to 300 nm, and more preferably 20 nm to 100 nm.

Further, a shape of a magnetic nanoparticle according to the present invention is not particularly limited, and may be, for instance, spherical, linear, cylindrical, flat, or any combination thereof.

Examples of the rare earth metal in the present invention include Sc, Y, La, Ce, Pr, Nd, Pm, Sm, Eu, Gd, Tb, Dy, Ho, Er, Tm, Yb, and Lu, preferably lanthanum metals, such as La, Ce, Pr, Nd, Pm, Sm, Eu, Gd, Tb, Dy, Ho, Er, Tm, Yb, and Lu, and more preferably La and Nd, but the present invention is not limited thereto.

Examples of the divalent metal according to the present invention include Be, Mg, Ca, Sr, Ba, Ra, Pb, V, Nb, Ta, Zn, Cd, and Hg, preferably alkali earth metals, such as Be, Mg, Ca, Sr, Ba, and Ra; and Pb, and more preferably Sr, Ba, Ca, and Pb, but the present invention is not limited thereto.

Examples of the transition metal oxide in the present invention include oxides of at least one metal selected from the group consisting of Ti, V, Cr, Mn, Fe, Co, Ni, Cu, Zn, Zr, Nb, Mo, Tc, Ru, Rh, Pd, Ag, Cd, Hf, Ta, W, Re, Os, Ir, Pt, Au, and Hg, and preferably manganese oxide, but the present invention is not limited thereto.

The magnetic nanoparticle may comprise 0.5 to 1 molar fraction of the rare earth metal and 0.01 to 0.5 molar fraction of the divalent metal, relative to 1 molar fraction of the transition metal oxide, but the present invention is not limited thereto.

In the magnetic nanoparticle of the present invention, a Curie temperature of the magnetic nanoparticle may be adjusted to the range of −80° C. to 41° C. by controlling the molar fraction of each component within the above-described ranges.

The magnetic nanoparticle of the present invention may form a structured body together with another material for an additional function. Types of the structured body are not particularly limited, and may be, for instance, a core-shell structure, a dumbbell structure, a cluster structure, a thin layer structure, an alloy structure, a multi-layered nanowire, or any combination thereof. Herein, the other materials constituting the structured body together with the magnetic nanoparticle may be a silica, a ceramic material, an organic material, a metallic material, a magnetic material, a polymer, or a semiconductor material, depending on the purpose of use, but the present invention is not limited thereto.

In the case of the core-shell structure in the present invention, a magnetic nanoparticle according to the present invention may form a core part while the above other material forms a shell part surrounding the core part. Alternatively, the above other material may form a core part while a magnetic nanoparticle according to the present invention forms a shell part. Herein, the shell part of the core-shell structure may have pores, and thus it can be a porous core-shell form. In the case of the porous core-shell in the present invention, a drug can be supported thereon, and thus it can be used as a drug delivering body.

In the case of the dumbbell structure in the present invention, one part of the dumbbell may be formed with a magnetic nanoparticle according to the present invention, while the other part is formed with another material, such as a magnetic material, a metallic material, a polymer, a ceramic and semiconductor material, depending on the purpose of use.

In the case of the multi-layered nanowire structure in the present invention, the nanowire may have a multi-layered structure wherein a magnetic nanoparticle according to the present invention and another material, such as gold (Au), are alternately formed.

In the case of the thin layer structure in the present invention, a magnetic nanoparticle according to the present invention may form a thin layer, and a layered structure can be formed with the thin layer made of a magnetic nanoparticle according to the present invention and another thin layer made of another material.

Furthermore, the structured body consisting of a complex combination of the above structures, such as a structured body wherein one-dimensional nanowire structures are projected from a thin layer structure or a structured body wherein spherical nanoparticles are attached to a nanowire, may be used depending on the purpose of use.

Another aspect of the present invention concerns methods for preparing a magnetic nanoparticle according to the present invention, comprising (a) a step of reducing a precursor of the rare earth metal, a precursor of the divalent metal, and a precursor of the transition metal oxide, thereby forming a magnetic nanoparticle; and (b) a step of heat treating the magnetic nanoparticle.

For preparing a magnetic nanoparticle according to the present invention, a step of dissolving the precursor of the rare earth metal, the precursor of the divalent metal, the precursor of the transition metal oxide, and a reducing agent in a solvent, heating to a temperature in the range of 80° C. to 130° C., and uniformly mixing for 1 to 2 hours at said temperature may be conducted prior to step (a).

In the present invention, types of the precursor of the rare earth metal are not particularly limited, and include the aforementioned rare earth metals as well as anything that can become the rare earth metal by reduction through an oxidation-reduction reaction. In the present invention, examples of the precursor of the rare earth metal include lanthanum acetylacetonate (La(acac)₂) and lanthanum nitrate(La(NO₃)₃6H₂O), preferably lanthanum acetylacetonate, but the present invention is not limited thereto.

In the present invention, types of the precursor of the divalent metal are not particularly limited, and include the aforementioned divalent metals as well as anything that can become the divalent metal by reduction through an oxidation-reduction reaction. In the present invention, examples of the precursor of the divalent metal include strontium acetylacetonate (Sr(acac)₃) and strontium acetate (Sr(CH₃COO)₂), preferably strontium acetylacetonate, but the present invention is not limited thereto.

In the present invention, types of the precursor of the transition metal oxide are not particularly limited, and include the aforementioned transition metals as well as anything that can become the transition metal oxide by reduction through an oxidation-reduction reaction. In the present invention, examples of the precursor of the transition metal oxide include manganese acetylacetonate (Mn(acac)₃) and manganese acetate (Mn(CH3COO)₂.4H₂O), preferably manganese acetylacetonate, but the present invention is not limited thereto.

In the present invention, molar fractions of the precursor of the rare earth metal, the precursor of the divalent metal, and the precursor of the transition metal oxide are the same as described above.

In the present invention, the reducing agent helps to reduce each of the precursor of the rare earth metal, the precursor of the divalent metal, and the precursor of the transition metal oxide through an oxidation-reduction reaction so that the rare earth metal, the divalent metal, and the transition metal oxide can be agglomerated into a single nanoparticle.

In the present invention, types of the reducing agent are not particularly limited, and anything can be used without limitation as long as it can reduce all of the precursor of the rare earth metal, the precursor of the divalent metal, and the precursor of the transition metal oxide. In the present invention, examples of the reducing agent include 1,2-hexadecanediol, but the present invention is not limited thereto.

In the present invention, a content of the reducing agent is not particularly limited, and can be properly selected within the scope being able to reduce all of the precursor of the rare earth metal, the precursor of the divalent metal, and the precursor of the transition metal oxide.

In the present invention, types of the solvents are not particularly limited, and anything can be used without limitation as long as it can dissolve the precursor of the rare earth metal, the precursor of the divalent metal, the precursor of the transition metal oxide, and the reducing agent. In the present invention, examples of the solvent include alkylethers having an alkyl group with 1 to 12 carbon atoms, arylethers having an aryl group with 6 to 18 carbon atoms, aralkylethers with 7 to 21 carbon atoms, and alkenylethers having an alkenyl group with 2 to 12 carbon atoms, but the present invention is not limited thereto.

In the present invention, a content of the solvent is not particularly limited, and can be properly selected within the scope of being able to disolve all of the precursor of the rare earth metal, the precursor of the divalent metal, the precursor of the transition metal oxide, and the reducing agent.

In the present invention, when the heating temperature is less than 80° C. in the preparation step of the mixed solution, the mixing of the components in a solvent may not be uniform, and when exceeding 130° C., the precursor or reducing agent may react in advance. Further, uniform mixing of each component in the mixed solution can be achieved by controlling the time for which the heating temperature is maintained within the above-described range.

In the preparation step of the mixed solution of the present invention, a surfactant may be further dissolved in a solvent along with the precursor of the rare earth metal, the precursor of the divalent metal, the precursor of the transition metal oxide, and the reducing agent.

In a case in which the surfactant is further dissolved in the preparation step of the mixed solution of the present invention, dispersibility of the magnetic nanoparticle in an aqueous solution as well as an affinity to a biological control agent can be increased.

In the present invention, types of the surfactant are not particularly limited, and any material can be used as long as it shows amphipathy. In the present invention, examples of the surfactant include polyalkyleneglycol, polyetherimide, polyvinylpyrrolidone, hydrophilic vinyl polymer, and copolymers of at least two of the aforementioned, but the present invention is not limited thereto.

In the present invention, when the copolymer is used, the copolymer can preferably be a block copolymer of polyethylene glycol (PEG)-polypropylene glycol (PPG)-polyethylene glycol (PEG) or a block copolymer of polyethylene oxide (PEO)-polypropylene oxide (PPO)-polyethylene oxide (PEO).

In a method for preparing a magnetic nanoparticle in the present invention, a step of reducing the precursor of the rare earth metal, the precursor of the divalent metal, and the precursor of the transition metal can be performed after preparing the mixed solution, as described above. The precursor components and the reducing agent contained in the mixed solution undergo an oxidation-reduction reaction such that the reducing agent are oxidized while the precursors are reduced to become the rare earth metal, the divalent metal, and the transition metal oxide.

In particular, a step of the reduction in the present invention may be performed by heating the mixed solution to a temperature in the range of 220° C. to 300° C., and maintaining the temperature for 1 to 2 hours. When the heating temperature is less than 220° C. in the reduction step, the oxidation-reduction reaction between the precursor components and the reducing agent may not be sufficient, and when exceeding 300° C., agglomeration of the nanoparticles may occur. Further, a smooth reduction of each precursor component can be achieved by controlling the time for which the heating temperature is maintained within the above-described range.

The method for preparing the magnetic nanoparticle of the present invention may be performed by a step of reducing each precursor component contained in the mixed solution to the rare earth metal, the divalent metal, and the transition metal oxide, and forming the magnetic nanoparticle by cooling it.

When each precursor component contained in the mixed solution is reduced to the rare earth metal, the divalent metal, and the transition metal oxide, and cooled as described above, the rare earth metal, the divalent metal, and the transition metal oxide may agglomerate during the cooling process, thereby forming nano-sized particles. In the present invention, the cooling temperature is not particularly limited and can be any temperature at which the nano-sized particles can be formed, and the cooling can preferably be conducted to a room temperature.

In the present invention, the methods for cooling the mixed solution are not particularly limited, and any conventional means in the art can be used without limitation.

In the method for preparing the magnetic nanoparticle of the present invention, step (a) can be performed under an inert gas atmosphere, such as an argon gas atmosphere. Unexpected oxidation of the precursor components or the magnetic nanoparticle can be prevented by performing step (a) under an inert gas atmosphere.

The method for preparing the magnetic nanoparticle of the present invention may further comprise a step of washing the magnetic nanoparticle formed in step (a) using centrifugation and magnetic separation after step (a).

In particular, after step (a), anhydrous ethanol can be added to the magnetic nanoparticle, and centrifugation and magnetic separation may be performed to remove remaining precursor components and reducing agent, thereby separating the magnetic nanoparticle only.

The method for preparing the magnetic nanoparticle of the present invention may comprise (b) a step of heat treating the magnetic nanoparticle. In the method for preparing the magnetic nanoparticle of the present invention, crystallinity of the magnetic nanoparticle can be increased by performing step (b), thereby enabling preparation of the magnetic nanoparticle having a Curie temperature within the range of 0° C. to 41° C.

In the method for preparing the magnetic nanoparticle of the present invention, step (b) may be performed by heating the magnetic nanoparticle to a temperature in the range of 300° C. to 1000° C. in a heating furnace, and maintaining the temperature for 1 to 13 hours. Types of the heating furnace are not particularly limited, and any means which is conventionally used in the art can be used. In the present invention, an exemplary heating furnace is a ceramic container, but the present invention is not limited thereto. When the heating temperature in the heat treatment step is less than 300° C., a heat treatment effect may not be sufficient, and when exceeding 1000° C., a production cost may increase due to excessive energy consumption. In addition, a time for which the heating temperature is maintained is preferably 2 to 12 hours, and by controlling as such, crystallinity of the magnetic nanoparticle can be increased.

In the method for preparing the magnetic nanoparticle of the present invention, step (b) may be performed in the heating furnace filled with an inert gas, such as argon gas and nitrogen gas, in order to control a degree of oxidation of the magnetic nanoparticle.

In addition, in the method for preparing the magnetic nanoparticle of the present invention, step (b) may be performed in a heating furnace in which an external magnetic field is applied in order to control magnetic properties of the magnetic nanoparticle. Types of the external magnetic field are not particularly limited, any magnetic field which is conventionally used in the art can be used without limitation, and also, a strength of the external magnetic field can be properly selected according to requirements.

The method for preparing the magnetic nanoparticle of the present invention may further comprise, prior to step (b), a step of coating the magnetic nanoparticle with a coating material in order to prevent calcination of the magnetic nanoparticle caused by a performance of step (b). Types of the coating material to coat the magnetic nanoparticle are not particularly limited, and preferably a ceramic material, or a semiconductor material such as zinc oxide, magnesium oxide or aluminum oxide, can be used.

In the present invention, the methods for coating the magnetic nanoparticle with the coating material are not particularly limited, any means which is conventionally used in the art can be used, but preference is given to use of thermal decomposition.

When the step of coating the magnetic nanoparticle with the coating material prior to step (b) is intended to be performed, after step (b), a treatment with an acidic or basic solution may be conducted to remove the coating material covering the magnetic nanoparticle, and after washing, the magnetic nanoparticle according to the present invention can be separated using a method such as centrifugation.

The method for preparing the magnetic nanoparticle of the present invention may further comprise, prior to step (b), a step of filling the magnetic nanoparticle in a nano-template as another means to prevent calcination of the magnetic nanoparticle caused by a performance of step (b). When the magnetic nanoparticle prepared in step (a) is filled in a nano-template and introduced into a heating furnace where the heat treatment is conducted, calcination of the magnetic nanoparticle during the heat treatment can be prevented. The method for filling the magnetic nanoparticle prepared in step (a) in the nano-template may be, for instance, the method described in Korean patent application No. 10-2004-0084468.

When the step of filling the magnetic nanoparticle in the nano-template prior to step (b) is performed as described above, after step (b), the nano-template can be dissolved using a chromic acid solution or a sodium hydroxide solution, thereby extracting the magnetic nanoparticle only.

The method for preparing the magnetic nanoparticle of the present invention may further comprise a process of separating a partially calcinated magnetic nanoparticle with a laser treatment or an ultrasonic wave treatment in order to remove the partially calcinated magnetic nanoparticle which may be produced in step (b).

Still another aspect of the present invention concerns nanocomposites comprising a magnetic nanoparticle according to the present invention; and a biological control agent attached to a surface of the magnetic nanoparticle.

The details as to the magnetic nanoparticle to be contained in the nanocomposite of the present invention are the same as described above.

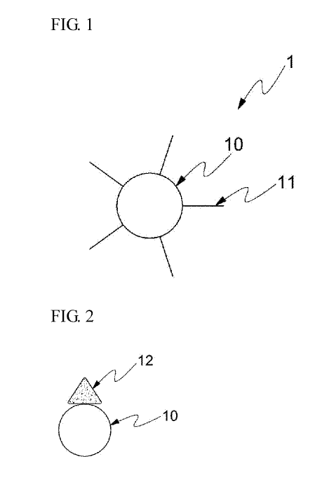

The appended FIG. 1 illustrates a nanocomposite according to one embodiment of the present invention. As illustrated in FIG. 1, a nanocomposite (1) of the present invention may comprise a magnetic nanoparticle (10) and a biological control agent (11) which is attached to a surface of the magnetic nanoparticle (10).

In the present invention, types of the biological control agent attached to a surface of the magnetic nanoparticle are not particularly limited, and preferably an antigen, an antibody, a protein, or a biocompatible polymer can be used.

In the present invention, types of the antigen, the antibody, and the protein are not particularly limited, and anything can be used without limitation as long as it can be conventionally used for target substance detection.

Introducing the antigen, the antibody, and the protein in the surface of a magnetic nanoparticle according to the present invention can be performed by methods well-known in the art. In the present invention, for instance, the antigen, the antibody, and the protein can be introduced by coating gold (Au) on a surface of a magnetic nanoparticle according to the present invention, and then introducing thiol groups on a surface of the gold coating, or the antigen, the antibody, and the protein can be introduced by attaching a biocompatible polymer on a surface of a magnetic nanoparticle according to the present invention by a method to be described below, and then bonding a functional group existing on an end part of the biocompatible polymer with a particular functional group.

The antigen, the antibody, and the protein attached to a surface of a magnetic nanoparticle according to the present invention may be used for detection and separation of target substance, such as detection and quantification of a target protein.

In the present invention, the biocompatible polymer attached to a surface of the magnetic nanoparticle can increase dispersibility of the magnetic nanoparticles in aqueous solution and affinity to the biological control agent.

In the present invention, types of the biocompatible polymer are not particularly limited, and any material can be used as long as it shows amphipathy. In the present invention, examples of the biocompatible polymer include polyalkyleneglycol, polyetherimide, polyvinylpyrrolidone, hydrophilic vinyl polymer, and copolymers of at least two of the aforementioned, but the present invention is not limited thereto.

In the present invention, when a copolymer is used as the biocompatible polymer, the copolymer can preferably be a block copolymer of polyethylene glycol (PEG)-polypropylene glycol (PPG)-polyethylene glycol (PEG) or a block copolymer of polyethylene oxide (PEO)-polypropylene oxide (PPO)-polyethylene oxide (PEO).

In the present invention, the method for introducing the biocompatible polymer to a surface of a magnetic nanoparticle according to the present invention is not particularly limited, and for instance, the magnetic nanoparticles to whose surface the biocompatible polymer is attached may be prepared, in step (a) of the method for preparing a magnetic nanoparticle according to the present invention, by dissolving the biocompatible polymer along with the precursor of the rare earth metal, the precursor of the divalent metal, the precursor of the transition metal oxide, and the reducing agent, thereby preparing a mixed solution, and performing step (b) in the same manner.

Upon preparing the nanocomposite of the present invention, a stabilizer, such as oleylamine (C₉H₁₈═C₉H₁₇NH₂) and oleic acid (C₉H₁₈═C₈H₁₅COOH), may be added to a solvent.

Yet another aspect of the present invention concerns a composition for target substance detection comprising a magnetic nanoparticle according to the present invention, or a nanocomposite according to the present invention, and a magnet-antibody composite.

The details of the magnetic nanoparticle or the nanocomposite contained in the composition for target substance detection of the present invention are the same as described above.

A detection means may be attached to a surface of the magnetic nanoparticle, or the nanocomposite to be contained in the composition for target substance detection of the present invention,

The appended FIG. 2 illustrates the magnetic nanoparticle of the present invention to whose surface a detection means is attached. As illustrated in FIG. 2, a detection means (12) is attached to a surface of the magnetic nanoparticle (10) of the present invention, and this can be used as the composition for target substance detection.

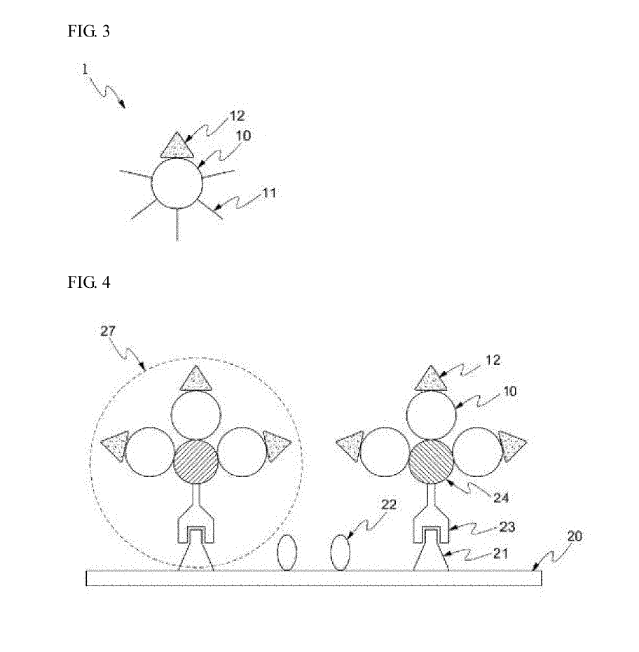

The appended FIG. 3 illustrates the nanocomposite of the present invention to whose surface a detection means is attached. As illustrated in FIG. 3, a detection means (12) is attached to a surface of the nanocomposite (1) of the present invention, and this can be used as the composition for target substance detection.

According to one embodiment of the present invention, the composition for target substance detection may be used for detecting a particular antigen, such as a particular protein or a particular cell, or an amount thereof, like in an ELISA method or a Western blot method.

The composition for target substance detection of the present invention can form a bond with the target substance by an antibody in the magnet-antibody composite through an antigen-antibody reaction when the target substance is present.

In particular, when a target substance, an antigen, is fixed on a substrate and the composition for target substance detection of the present invention comprising the magnet-antibody composite which may cause an antigen-antibody reaction with the target substance is covered thereon, a single composite consisting of target substance-antibody-magnet can be formed through an antigen-antibody reaction of the target substance and the antibody part of the magnet-antibody composite.

In addition, when the magnetic nanoparticle or the nanocomposite to whose surface a detection means is attached is maintained at a temperature equal to or above a Curie temperature, it loses magnetic properties, and thus is not agglomerated but rather uniformly dispersed in the composition. However, when the composite consisting of target substance-antibody-magnet is formed, the magnetic nanoparticle or the nanocomposite returns to a ferromagnet by controlling the temperature to be equal to or less than a Curie temperature, and thus agglomeration may occur due to an attractive force with a magnet part of the composite consisting of target substance-antibody-magnet.

Herein, when the composition for target substance detection is washed, an individual magnetic nanoparticle or nanocomposite which is not agglomerated with the composite consisting of target substance-antibody-magnet may be removed.

Accordingly, a massive composite consisting of target substance-antibody-magnet-magnetic nanoparticle-detection means, or a massive composite consisting of target substance-antibody-magnet-nanocomposite-detection means can be formed.

The detection means in the massive composite can transmit a particular signal depending on its type, thus enabling detection of a target substance. In a part where the target substance is present, the particular signal can be observed, while in a part where the target substance is not present, the particular signal cannot be observed.

The appended FIG. 4 is a schematic diagram showing a process of detecting a target substance using a composition for target substance detection according to one embodiment of the present invention. As illustrated in FIG. 4, when a target substance (21) is fixed on substrate (20), the target substance (21) and an antibody (23) undergo an antigen-antibody reaction, thereby forming the composite consisting of target substance (21)-antibody (23)-magnet (24). Herein, when the temperature is maintained at equal to or above a Curie temperature of the magnetic nanoparticle of the present invention, a magnetic nanoparticle (10) to whose surface a detection means (12) is attached may lose magnetic properties, and thus agglomeration of the magnetic nanoparticles does not occur and a well-dispersed form is achieved. However, when the temperature is lowered to less than a Curie temperature of the magnetic nanoparticle of the present invention, the magnetic nanoparticle (10) re-gains ferromagnetic properties and can be agglomerated due to an attractive force with the magnet (24). Thus, a massive composite (27) consisting of target substance (21)-antibody (23)-magnet (24)-magnetic nanoparticle (10)-detection means (12) fixed on substrate (20) can be formed. When a container comprising the composition for target substance detection is washed, components other than the massive composite (27), such as the magnetic nanoparticle to which a detection means is attached, can be removed. In the case of the massive composite (27) fixed on a substrate as described above, a particular signal can be transmitted through the detection means (12), and thus presence of the target substance can be confirmed. Furthermore, in a case of impurities (22) which cannot undergo an antigen-antibody reaction with an antibody (23), the particular signal cannot be observed therein as the massive composite (27) cannot be formed.

The composition for target substance detection of the present invention may form the massive composite through specific bonding with a target substance and control of the magnetic properties of the magnetic nanoparticle, thereby increasing a ratio of signal to noise (signal purification). In other words, the composition for target substance detection of the present invention can increase both specificity and sensitivity to the target substance.

In the composition for target substance detection of the present invention, the detection means is not particularly limited, and any detection means may be used without limitation as long as it can be used in imaging of a living body. In the present invention, examples of the detection means include a fluorescent material and a quantum dot, but the present invention is not limited thereto.

In the present invention, when the fluorescent material is used as the detection means, confirmation of a target substance, quantitative analysis, and separation can be performed through a fluorescent image. In the present invention, types of the fluorescent material are not particularly limited, and examples thereof include rhodamine and its derivatives, fluorescein and its derivatives, coumarin and its derivatives, acridine and its derivatives, pyrene and its derivatives, erythrosine and its derivatives, eosin and its derivatives, and 4-acetamido-4′-isothiocyanatostilbene-2,2′-disulfonic acid. Further particular examples of the fluorescent material which can be used in the present invention are as follows.

Examples of the rhodamine and its derivatives include 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101, sulfonyl chloride derivatives of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives, Alexa derivatives, Alexa-350, Alexa-488, Alexa-547, and Alexa-647;

examples of the fluorescein and its derivatives include 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate, QFITC (XRITC), fluorescamine, IR144, IR1446, malachite green isothiocyanate, 4-methylumbelliferone, ortho-cresolphthalein, nitrotyrosine, pararosaniline, phenol red, B-phycoerythrin, and o-phthaldialdehyde;

examples of the coumarin and its derivatives include coumarin, 7-amino-4-methylcoumarin (AMC, coumarin 120), 7-amino-4-trifluoromethylcoumarin (coumarin 151), cyanocin, 4′-6-diamidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentacetate, 4-(4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL), and 4-dimethylaminophenylazophenyl-4′-isothiocyanate (DABITC);

examples of the acridine and its derivatives include acridine, acridine isothiocyanate, 5-(2′-aminoethyl)aminonaphthalene-1-sulfonic acid (EDANS), 4-amino -N-[3-vinylsulfonyl)phenyl]naphthalimide-3,5disulfonate (LuciferYellow VS), N-(4-anilino-1-naphthyl)maleimide, anthranilamide, and Brilliant Yellow;

examples of the pyrene and its derivatives include pyrene, pyrene butyrate, succinimidyl 1-pyrene butyrate, and Reactive Red 4 (Cibacron Brilliant Red 3B-A);

examples of the erythrosine and its derivatives include erythrosin B, erythrosin isothiocyanate, and ethidium;

examples of the eosin and its derivatives include eosin, and eosin isothiocyanate; and
4-acetamido-4′-isothiocyanatostilbene-2,2′disulfonic acid.

In the present invention, when a quantum dot is used as the detection means, detection of a target substance, quantitative analysis and separation can be performed through a fluorescent image. The quantum dot may have a structure consisting of a center part, a shell part surrounding the center part, and a polymer coating layer coated on the shell part. In the present invention, types of the quantum dot are not particularly limited, and anything may be used without limitation as long as it has biocompatibility and can be used for imaging of a living body. As the components consisting of the center part of the quantum dot, cadmium selenide (CdSe), cadmium telluride (CdTe), cadmium sulfide (CdS), zinc selenide (ZnSe), zinc oxide (ZnO), or zinc sulfide (ZnS) can be mainly used, but the present invention is not limited thereto.

In the magnet-antibody composite to be contained in the composition for target substance detection of the present invention, types of the magnet are not particularly limited, and anything can be used without limitation as long as it has magnetic properties. In the present invention, for example, a magnetic nanoparticle according to the present invention or a conductive material can be used as the magnet, but the present invention is not limited thereto.

In the present invention, types of the conductive material are not particularly limited, and examples thereof are a metallic material, a magnetic material, and a magnetic alloy. Further examples of the conductive material which can be used in the present invention are as follows.

Examples of the metallic material include Pt, Pd, Ag, Cu, and Au, examples of the magnetic material include Co, Mn, Fe, Ni, Gd, and Mo, and examples of the magnetic alloy include CoCu, CoPt, FePt, CoSm, NiFe, and NiFeCo, but the present invention is not limited thereto.

In addition, in the magnet-antibody composite of the present invention, types of the antibody are not particularly limited, and anything may be used without limitation as long as it can be bonded to the target substance described below through an antigen-antibody reaction.

Types of the target substance to be detected using the composition for target substance detection of the present invention are not particularly limited, and can be, for instance, at least one selected from the group consisting of a protein, a DNA, and a RNA. In the present invention, types of the protein, the DNA, and the RNA are not particularly limited, and examples thereof can be made to a tumor marker or a bio-marker which is conventionally used in the art.

In the present invention, a protein, the target substance, can be at least one selected from the group consisting of prostate specific antigen (PSA), carcinoembryonic antigen (CEA) MUC1, alpha fetoprotein (AFP), carbohydrate antigen 15-3 (CA 15-3), carbohydrate antigen 19-9 (CA 19-9), carbohydrate antigen 125 (CA 125), free prostate specific antigen (PSAF), prostate specific antigen- a 1-anticymotrypsin comple (PSAC), prostatic acid phosphatase (PAP), human thyroglobulin (hTG), human chorionic gonadotropin beta (HCGb), ferritin (Ferr), neuron specific enolase (NSE), interleukin 2 (IL-2), interleukin 6 (IL-6), beta 2 macroglobulin (B2M), and alpha 2 macroglobulin (A2M), but the present invention is not limited thereto.

PSA, PSAF, PSAC, A2M, and PAP are useful tumor markers in the selection of prostate cancer, CEA is a useful tumor marker in the selection of gastrointestinal cancer as a glycoprotein, MUC1 is a tumor marker expressed in ovarian cancer, breast cancer, myeloma, colon cancer, uterine cancer, pancreatic cancer, rectal cancer, and lung cancer, CA 15-3 is a tumor marker expressed in lung cancer, pancreatic cancer, breast cancer, ovarian cancer, and liver cancer, CA 19-9 is a tumor marker expressed in lung cancer, ovarian cancer, liver cancer, and colon cancer, CA 125 is a tumor marker expressed in lung cancer, pancreatic cancer, breast cancer, ovarian cancer, liver cancer, colon cancer, and uterine cancer, hTG is a tumor marker expressed in thyroid cancer and Wilm's tumor, HCGb is a tumor marker expressed in lung cancer, pancreatic cancer, kidney cancer, ovarian cancer, liver cancer, brain cancer, and bladder cancer, Ferr is a tumor marker expressed in lung cancer and brain cancer, NSE is a tumor marker expressed in lung cancer, thyroid cancer, and Wilm's cancer, IL-2 is a tumor marker expressed in kidney cancer, and multiple myeloma, IL-6 is a tumor marker expressed in kidney cancer, breast cancer, ovarian cancer, and multiple myeloma, and B2M is a tumor marker expressed in kidney cancer, ovarian cancer, prostate cancer, and multiple myeloma.

In the present invention, the DNA, the RNA, and the target substance are not particularly limited, and any gene may be used without limitation as long as it is the gene of a virus which invokes infectious disease. In the present invention, examples of the DNA and the RNA include a gene of AIDS virus, a gene of hepatitis B virus, a gene of hepatitis C virus, a gene of malaria virus, a gene of novel swine-origin influenza virus, or a gene of syphilis virus, but the present invention is not limited thereto.

A further aspect of the present invention concerns a method for obtaining an image of a living body or specimen, comprising a step of administering a composition for target substance detection according to the present invention to the living body or specimen; and a step of sensing a signal transmitted by the nanocomposite from the living body or specimen, thereby obtaining the image.

In the present invention, an expression “specimen” may denote a tissue or cell which is separated from the subject to be diagnosed. Further, the step of administering the composition for target substance detection of the present invention to a living body or specimen can be performed through a path which is conventionally used in the domain of pharmaceuticals, preferably parenteral administration, such as an administration through an intravenous, intraabdominal, intramuscular, subcutaneous, or topical path. In the step of obtaining the image of the present invention, magnetic resonance imaging (MRI) and optical imaging are preferably used in order to sense the signal transmitted by a fluorescent material or quantum dot.

In the present invention, the expression “magnetic resonance imaging apparatus” may denote an imaging apparatus into which a living body is introduced, energy is absorbed in an atomic nucleus, such as hydrogen, in a tissue of the living body by electromagnetic irradiation at a particular frequency so that a high-energy state is created, then the energy of the atomic nucleus, such as hydrogen, is released after irradiation, and the energy is transformed into a signal which is in turn processed to yield an image. In the present invention, a type of the magnetic resonance imaging apparatus is not particularly limited, and can be, for instance, a T2 spin-spin relaxation magnetic resonance imaging apparatus, but the present invention is not limited thereto. Meanwhile, in the present invention, a co-focal microscope, a fluorescence microscope or an optical equipment for a living body can be used for imaging, but the present invention is not limited thereto.

In the method for obtaining an image of a living body or specimen according to the present invention, the composition for target substance detection is administered to a living body or specimen, and thereby the composite consisting of target substance-antibody-magnet can be formed by an antigen-antibody reaction with a particular antigen which is a target substance. Thereafter, when the temperature of a magnetic nanoparticle according to the present invention is maintained to be below a Curie temperature using a magnetocaloric effect, a massive composite of target substance-antibody-magnet-nanocomposite-detection means can be formed through ferromagnetic properties of the magnetic nanoparticle as described above. In this case, the massive composites comprising a detection means are distributed in a high concentration around a particular antigen, and thus an amplified image signal can be easily obtained. The magnetocaloric effect is a phenomenon of gradually getting colder or hotter due to a quick transition of a magnetization status of the magnetic material within an external magnetic field, and is well-known in the art.

Hereinafter, Examples of the present invention will be described in detail. However, the present invention is not limited to Examples disclosed below, but may be implemented in various forms. The following Examples are described in order to enable those of ordinary skill in the art to embody and practice the present invention.

### EXAMPLES

### Example 1

A magnetic nanoparticle of the present invention was prepared by an improved nano-emulsion method based on thermal decomposition as described below.

(1) Preparation of a mixed solution

0.45 mmol of lanthanum acetylacetonate (La(acac)₃, available from Aldrich), a precursor of rare earth metal, 0.15 mmol of strontium acetylacetonate (Sr(acac)₂, available from Aldrich), a precursor of divalent metal, 0.6 mmol of manganese acetylacetonate (Mn(acac)₃, available from Aldrich), a precursor of transition metal oxide, and 0.1294 g of 1,2-hexadecanediol (available from Aldrich), a reducing agent, were introduced to a container comprising 15 ml of dioctylether (available from Wako) under an argon gas atmosphere and dissolved. Thereafter, the solution was heated to 100° C. and uniformly stirred for 1.5 hours at 100° C. to result in the mixed solution.

(2) Reduction of the precursors contained in the mixed solution

Thusly-prepared mixed solution was heated to 280° C. and maintained for 1.5 hours at 280° C. to reduce lanthanum acetylacetonate, strontium acetylacetonate, and manganese acetylacetonate to lanthanum metal (La), strontium metal (Sr), and manganese oxide (MnO₃), respectively, through an oxidation-reduction reaction with 1,2-hexadecanediol.

(3) Formation of a magnetic nanoparticle

The mixed solution in which all the precursor components were reduced as described above was cooled down to room temperature, thereby forming a magnetic nanoparticle (LaSrMnO₃) in which lanthanum metal, strontium metal, and manganese oxide were agglomerated. An average diameter of the magnetic nanoparticle was about 30 nm.

(4) Washing the magnetic nanoparticle using centrifugation and magnetic separation

The formed magnetic nanoparticle was added to anhydrous ethanol, and washed with centrifugation and magnetic separation, thereby removing impurities.

(5) Heat treatment of the magnetic nanoparticle

The washed magnetic nanoparticle was introduced into a ceramic container, heated to 800° C., and maintained at 800° C. for 12 hours to perform heat treatment.

### Example 2

The nanocomposite illustrated in the appended FIG. 1 was prepared in the same manner as Example 1, except that, during the process of preparing the (1) mixed solution, 0.1576 g of a block copolymer of polyethylene glycol-polypropylene glycol-polyethylene glycol (available from Aldrich), a biocompatible polymer, was further dissolved in 15 ml, of dioctylether (available from Wako), the solvent.

### Experimental Example 1

In order to measure a shape of the magnetic nanoparticle prepared in Example 1, the magnetic nanoparticle prepared in Example 1 was dispersed in hexane and dropped on carbon-supported copper grids to prepare a sample for TEM measurement. Thereafter, TEM (Tecnai F20, available from FEI) and energy-dispersive X-ray spectroscopy (EDS) were used to observe the sample. The appended FIG. 5 is a picture of a high-resolution TEM of a magnetic nanoparticle according to one embodiment of the present invention. As illustrated in FIG. 5, a scale bar denotes 5 nm and the magnetic nanoparticle of Example 1 showed an average diameter of about 30 nm.

### Experimental Example 2

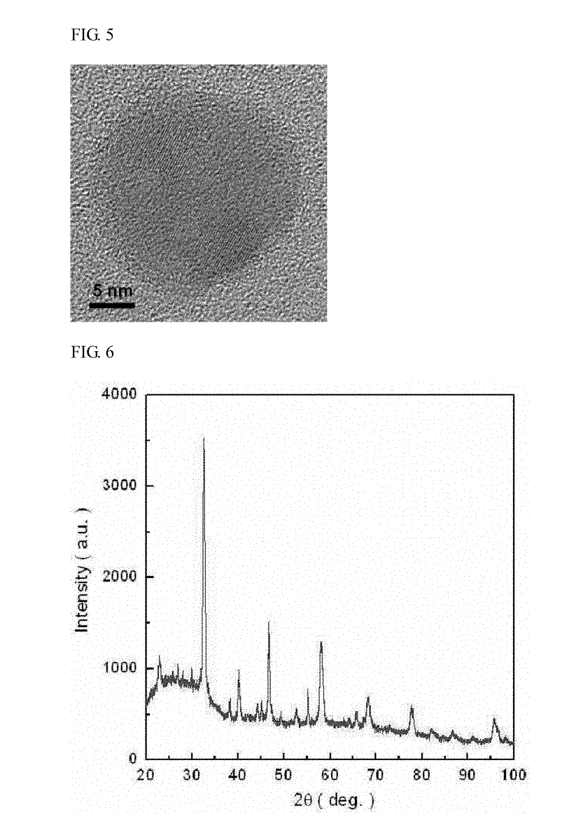

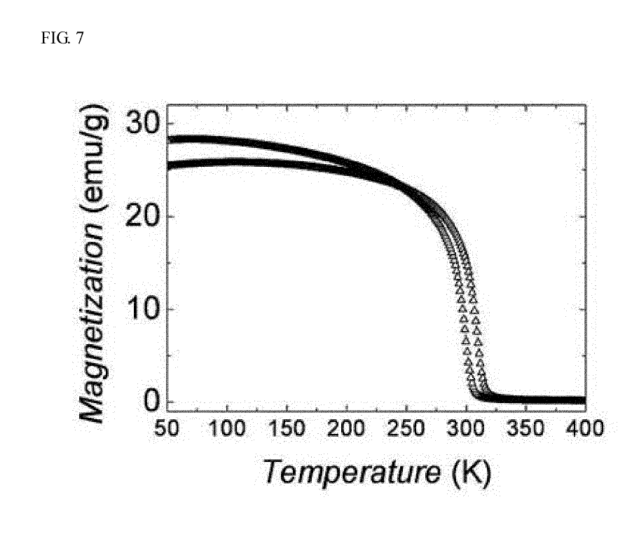

In order to analyze a structure of the magnetic nanoparticle prepared in Example 1, X-ray diffraction analysis of the sample prepared in Experimental Example 1 was performed using an X-ray diffractometer. The appended FIG. 6 is a graph showing an X-ray diffraction (XRD) pattern of a magnetic nanoparticle according to one embodiment of the present invention. As illustrated in FIG. 6, the magnetic nanoparticle of the present invention showed superior crystallinity.

### Experimental Example 3

In order to measure magnetic properties of the magnetic nanoparticle prepared in Example 1, a change of magnetization value of the sample prepared in Experimental Example 1 in accordance with temperature was measured using a vibrating sample magnetometer (VSM, VSM 7300, available from Lakeshore) and a physical property measurement system (PPMS, available from Quantum Design). The appended FIG. 7 is a graph of magnetization value versus temperature (M-T) of a magnetic nanoparticle according to one embodiment of the present invention at 100 Oe. As illustrated in FIG. 7, the magnetic nanoparticle (La_{0.75}Sr_{0.25}(MnO₃)₁) of the present invention comprising a rare earth metal, a divalent metal, and a transition metal oxide had a magnetization value of 0 at temperatures equal to and above 310 K (37° C.).

As the magnetic nanoparticle of the present invention has a Curie temperature within the temperature range of 0° C. to 41° C., the ferromagnetic and paramagnetic properties of the magnetic nanoparticle may be controlled within a biocompatible temperature range at which a biological control agent is not destroyed, and the temperature of the magnetic nanoparticle is adjusted to control the magnetic properties thereof such that the properties of the magnetic nanoparticle may be used only when ferromagnetic properties are required, such as in a case of signal amplification in detecting, separating, and delivering biological control agents. Accordingly, the magnetic nanoparticle of the present invention may minimize adverse effects of ferromagnetic properties, and may be used in the effective detection and separation of biological control agents.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A method for preparing a magnetic nanoparticle having a Curie temperature within the range of 0° C. to 41° C., and comprising a rare earth metal, a divalent metal, and a transition metal oxide;
comprising (a) a step of reducing a precursor of the rare earth metal, a precursor of the divalent metal, and a precursor of the transition metal oxide, thereby forming the magnetic nanoparticle; and (b) a step of heat treating the magnetic nanoparticle.

2. The method according to claim 1, further comprising, prior to step (a), a step of dissolving the precursor of the rare earth metal, the precursor of the divalent metal, the precursor of the transition metal oxide, and a reducing agent in a solvent, heating to a temperature in the range of 80° C. to 130° C., and uniformly mixing for 1 to 2 hours at said temperature.

3. The method according to claim 2, wherein the step of preparing the mixed solution further comprises dissolving a surfactant in the solvent along with the precursor of the rare earth metal, the precursor of the divalent metal, the precursor of the transition metal oxide, and the reducing agent.

4. The method according to claim 2, wherein the reduction is performed by heating the mixed solution to a temperature in the range of 220° C. to 300° C., and maintaining the temperature for 1 to 2 hours.

5. The method according to claim 2, wherein the formation of the magnetic nanoparticle is performed by cooling the mixed solution to room temperature

6. The method according to claim 1, further comprising, after step (a), a step of washing the magnetic nanoparticle using centrifugation and magnetic separation.

7. The method according to claim 1, wherein step (b) is performed by heating the magnetic nanoparticle to a temperature in the range of 300° C. to 1000° C., and maintaining the temperature for 1 to 13 hours.

8. The method according to claim 7, wherein step (b) is performed under an inert gas atmosphere.

9. The method according to claim 7, wherein step (b) is performed under an external magnetic field.

10. The method according to claim 1, further comprising, prior to step (b), a step of coating the magnetic nanoparticle with a ceramic material or a semiconductor material.

11. The method according to claim 1, further comprising, prior to step (b), a step of filling the magnetic nanoparticle in a nano-template.
